(19) United States
(12) Patent Application Publication
(10) Pub. No.: US 2010/0036228 A1
(43) Pub. Date: Feb. 11, 2010

(54) SUTURE SLEEVE AND A METHOD FOR IMPLANTING ONE OR MORE ELECTRICAL LEADS INTO A VEIN

(76) Inventors: Johan Eckerdal, Knivsta (SE); Rolf Hill, Jarfalla (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/531,719

(22) PCT Filed: Mar. 26, 2007
(86) PCT No.: PCT/SE07/00294

§ 371 Date: Sep. 17, 2009

(51) Int. Cl. A61B 5/04

(52) U.S. Cl. 600/375

(57) ABSTRACT

In a suture sleeve and implantation method for one or more implantable leads, the suture sleeve is adapted to be inserted into a vein to secure and protect the one or more leads from damage when a suture thread is positioned and tied around the vein in the region over the suture sleeve to secure the suture sleeve and prevent bleeding from the vein. The suture sleeve has two or more lead receiving through holes into each of which a medical implantable lead may be inserted. The suture sleeve is formed such that at least all of the holes except one are provided with sealing means, which easily can be broken or removed when inserting a lead into the hole such that, when inserted into a vein, bleeding is prevented from a through hole of the sleeve by the sealing means even if no lead is positioned in the hole. The invention also relates to a method for implanting one or more electrical leads into a vein.

### BACKGROUND OF THE INVENTION

1. Field of the Invention

The invention relates to a suture sleeve for one or more implantable leads, the suture sleeve being adapted to be inserted into a vein to secure and protect the one or more leads from damage when a suture thread is positioned and tied around the vein in the region over the suture sleeve to secure the suture sleeve and prevent bleeding from the vein, of the type having two or more lead receiving through holes, into each of which a medical implantable lead may be inserted.

The invention also relates to a method for implanting one or more electrical leads into a vein.

2. Description of the Prior Art

It is sometimes desirable to implant an electrical lead into a vein in an animal or human body, such that the lead extends out from the vein. This is the case e.g. when implanting a pacemaker into a body for monitoring and controlling the heart function. The pacemaker itself is implanted just under the skin in a pacemaker pocket at a suitable position, whereas one or more leads are inserted through an opening in the wall of a vein and pushed in until a distal end enters the heart where it can be attached to the heart wall.

However, the vein must be closed after lead insertion to avoid bleeding and the lead must be fixated in relation to the vein to eliminate the risk that the lead accidentally can be drawn out from its position. Avoiding bleeding is very important to prevent local hematoma since hematoma is considered as a major positive predictor for infection in the pacemaker pocket. This is accomplished by means of a suture thread, which is positioned around the vein and the lead in the area of the cut opening in the wall of the vein and which is tied around the vein and the lead. However, when closing the vein by tying the suture around the vein and the lead, there is a risk that the lead might get damaged by squeezing of the suture thread around the lead if the suture is made too tight. The suture stress will be concentrated to a small area and may cause intensive abrasion load to the lead body. On the other hand, if the suture is not tight enough, bleeding may occur and cause hematoma. If the suture is not combined with fixation of the lead elsewhere, there is also a risk for longitudinal lead movement and lead dislodgment if the suture is not sufficient tight.

To eliminate these risks it is known to use a so called suture sleeve, which is positioned around the lead in the area of the lead-through in the wall of the vein. In this way the suture sleeve will protect the lead from damage by the suture thread and the suture sleeve will fix the lead in a sufficient degree.

However, sometimes it is desirable to connect the pacemaker with the heart by means of two separate leads. In such a case it is common practice to tying the suture around the vein directly onto unprotected leads to prevent from bleeding. The leads are then fix separately some distance from the vein entrance by using the suture sleeves to prevent from longitudinal lead movement and lead dislodgement. This has the effect that the leads in the venous entrance, where they are unprotected from the suture thread, may become damaged.

U.S. Pat. No. 5,107,856 discloses a suture sleeve for two leads and another for three leads. The suture sleeve is formed as a flexible strip having two or three spaced apart lead receiving channels. During use, one lead is positioned in each of the lead receiving channels and then the flexible strip is wrapped around the leads. Finally, the suture sleeve is positioned in the cut opening in the vein and tied around by a suture thread.

However, there are several disadvantages with such a suture sleeve. For example, it is not possible to position and fixate only one or two leads in a suture sleeve, which is adapted for three leads, since then blood will leak out through the channel which has no lead positioned therein. I.e. when inserting two leads into a vein it is necessary to use a suture sleeve being adapted for exactly two leads, and when inserting three leads into the vein it is necessary to use a suture sleeve being adapted for exactly three leads. Accordingly, it is necessary to keep in stock several types of suture sleeves to be prepared for different applications. Moreover, the leads in question are very small having a cross sectional dimension of only about 2 mm, which has to effect that also the suture sleeve will be very small. This will have to result that it is very difficult to wrap around the leads properly with the suture sleeve, insert it through the opening in the vein and fixate the assembly by means of a suture thread in a correct position without losing the suture sleeve during the handling. With a suture sleeve of this kind it is also impossible to completely eliminate bleeding since, as is evident from the drawings in that patent, there will always remain some gaps between the leads and the suture sleeve in an assembled state.

### SUMMARY OF THE INVENTION

It is an object of the invention to overcome the disadvantages with prior art suture sleeves. More specifically it is an object to provide a suture sleeve for one or more electrical leads which is easy to handle and by means of which it is possible to effectively eliminate any bleeding independently of whether there are leads positioned in every through hole or not.

The invention also relates to a method for implanting one or more electrical leads into a vein of a human or animal body, having essentially the same object as above.

The basis of the invention is the insight that the above object may be achieved by means of a suture sleeve having two or more lead receiving through holes. At least all the holes except one are provided with sealing means, which are easy to break or remove when it is desirable to insert a lead into a specific hole. Accordingly, as long as no lead is inserted into a hole and the sealing means is intact, the sealing means prevents any blood leakage through that hole.

Within this general idea, the invention can be modified in many different ways. In a hereinafter described and in the drawings illustrated embodiment of the invention, the sealing means is in form of a thin membrane, of e.g. silicone, which easily can be broken. However, the sealing means also could be in any other suitable form, e.g. as a removable plug or the like in the hole. Normally, this is not a preferred embodiment, since small removable items in connection with an open cut in a body, could constitute a risk that the item is lost in the cut. The membrane and the plug or the like, could be manufactured as a separate detail and attached in the hole of the suture sleeve by means of for example an adhesive. However, it is preferred to make the sealing means in the same production step as the suture sleeve itself, e.g. by injection molding.

The through holes in the suture sleeve can be made as complete and unbroken holes, in which case the leads are inserted by displacing the leads in the axial direction of each hole. In such a case a sealing means in form of a membrane can be broken by means of the lead when it is inserted into the hole. A prerequisite for forming the suture sleeve in this way is that the lead is substantial isodiametric along its entire length.

However, in many cases the leads have a thickened portion, e.g. in their tip portions. For this reason, according to a hereinafter described embodiment, at least all of the holes except one are formed with a slot along the entire length of the hole and the suture sleeve is formed of an elastic material. In this way a lead can be inserted into a through hole by deflecting the material around the hole, for widening the slot, and inserting the lead sideways through the widened slot. It is, in such a case, favorable if the slot formed through holes, are each provided with a sealing means in form of a membrane, which will brake when deflecting the material around the hole and widening the slot. However, one of the through holes can, as in the described and illustrated embodiment, be unbroken and adapted to be pre-assembled onto a lead. In this way there is no risk of losing the suture sleeve during an implantation.

Moreover, the suture sleeve could be manufactured with an arbitrary number of holes, however at least two. Normally it is sufficient to manufacture the suture sleeve with three holes since this will cover most application ranges for pacemakers. Then it is possible to attach one, two or three electrical leads, as desired, in a secured and bleeding preventing way.

### BRIEF DESCRIPTION OF THE DRAWINGS

FIG. 1 is a perspective view of a suture sleeve according to the present invention.

FIG. 2 is an end view of the suture sleeve of FIG. 1.

FIG. 3 is an end view according to FIG. 2, with the material around a slot in one of the through holes deflected outwardly.

FIG. 4 is a perspective view showing the suture sleeve in accordance with the invention, with two electrical leads therein, inserted into an opening in a vein and tied around with a suture thread.

### DESCRIPTION OF THE PREFERRED EMBODIMENTS

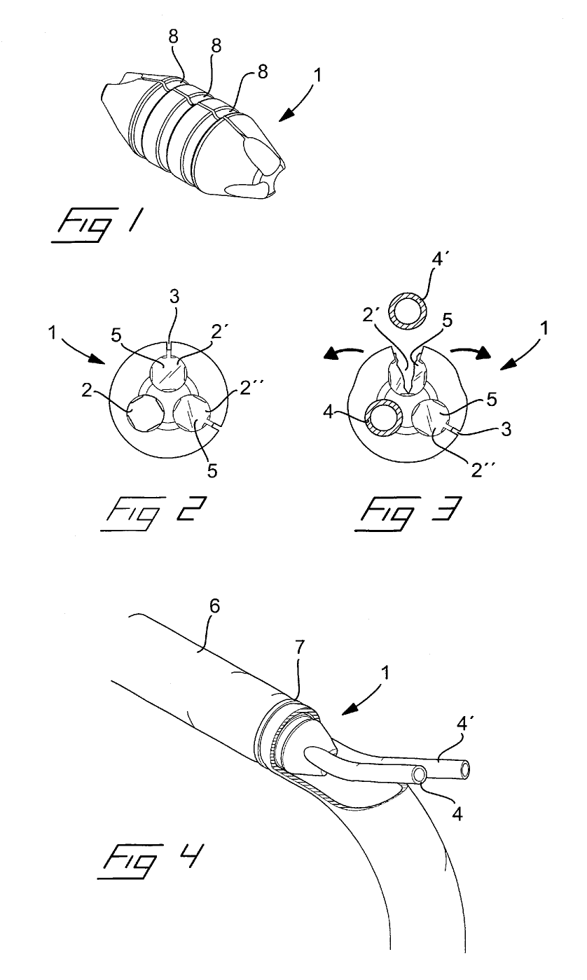

Reference is first made to FIGS. 1 and 2 of the drawings, in which a suture sleeve 1 according to the invention is shown in a perspective view and an end view, respectively. The suture sleeve has an elongated form with a generally circular cross section and is provided with a truncated conical shape in each end to facilitate introducing into an opening in a vein.

The suture sleeve comprises three through holes 2, 2′ and 2″ in its longitudinal direction. As evident from FIG. 2, one of the through holes 2 is unbroken, whereas the other through holes 2′ and 2″ are formed with a slot 3 in its longitudinal direction. The reason for forming the through holes in this way is that, as is illustrated in FIG. 3, the suture sleeve is adapted to be pre-assembled onto one electrical lead 4 such that the lead extends through the unbroken through hole 2. In this way the suture sleeve will be held safely on the lead without any risk for it to be lost during implantation. Subsequently one or two additional leads 4′ can optionally be mounted in the suture sleeve by, as is illustrated in FIG. 3, simply deflecting the material around the slot 3 such that the slot is widened and a lead can be inserted into the through hole 2′ sideways.

Each of the through holes 2′, 2″ is formed with a slot 3, and is provided with a sealing means in form of a thin membrane 5, which blocks the through hole. When mounting a lead into one of these through holes, the membrane will break as the slot is widened by deflecting the material around the slot, as is illustrated in FIG. 3.

FIG. 4 illustrates a situation where a suture sleeve 1 is partly inserted into an opening in a vein 6. Two leads 4, 4′ are inserted into the vein via a respective through hole 2, 2′ in the suture sleeve. Although the third through hole 2″ does not accommodate any lead, bleeding is prevented from this through hole owing to the fact that the membrane 5 still is intact in this hole. A suture thread 7 is positioned and tied around the vein 6 and the suture sleeve 1. In this way bleeding is prevented in the region between the suture sleeve and the wall of the vein. In order to improve the sealing and fixation between the suture sleeve and the vein, the suture sleeve is formed with circumferential grooves 8 around its outer periphery, as is best seen in FIG. 1, in which the suture thread 7 and vein tissue may sink into when tying the suture thread.

Although modifications and changes may be suggested by those skilled in the art, it is the intention of the inventors to embody within the patent warranted heron all changes and modifications as reasonably and properly come within the scope of their contribution to the art.

We claim as our invention:

1-14. (canceled)

15. A suture sleeve for one or more implantable medical leads, comprising:
a suture sleeve body configured for insertion into a vein;
said suture sleeve body comprising at least two through-holes each configured to receive a medical lead therein;
at least one of said through-holes having a seal therein, said seal being frangible or removable to allow insertion of a medical lead into that through-hole; and
said suture sleeve body being configured to be inserted into a vein to protect the medical leads therein from damage and being configured to be secured in the vein by a suture thread tied around said suture sleeve body to prevent bleeding from the vein, and said seal preventing bleeding from the through-hole containing the seal even when no lead is in that through-hole.

16. A suture sleeve as claimed in claim 15 wherein seal comprises a thin membrane.

17. A suture sleeve as claimed in claim 15 wherein said sleeve body is formed of an elastic material, and wherein each through-hole that comprises a seal therein has a slot proceeding in a longitudinal direction of that through-hole, the material around said slot being deflected for widening the slot and inserting a medical lead sideways into the through-hole through the widened slot, said seal being located in said slot and being frangible or removable upon opening and widening of the slot.

18. A suture sleeve as claimed in claim 15 wherein all of said through-holes comprise a seal.

19. A suture sleeve as claimed in claim 15 wherein said one of said through-holes without said seal is unbroken and is configured for pre-assembly on a medical lead.

20. A suture sleeve as claimed in claim 15 wherein said suture sleeve body has a generally circular cross-section.

21. A suture sleeve as claimed in claim 15 wherein said suture sleeve body comprises at least one circumferential groove proceeding around an exterior of the sleeve body and configured to receive suture thread therein.

22. A suture sleeve as claimed in claim 15 wherein said sleeve body has a truncated conical shape.

23. A method for implanting one or more electrical leads into a vein by means of a suture sleeve, which is adapted to secure and protect the one or more leads from damage when a suture thread is tied around the vein in the region over the suture sleeve to secure the suture sleeve and prevent bleeding from the vein, comprising the steps of:
providing a suture sleeve comprising two or more lead receiving through holes;
providing at least each hole except one with a sealing means, which easily can be broken or removed;
inserting one or more electrical leads into the suture sleeve and into the vein, while removing the sealing means in the respective hole; and
positioning the suture sleeve into the opening in the wall of the vein and tie a suture thread around the vein in the region of the suture sleeve.

24. A method according to claim 23, comprising the further step of:
providing a suture sleeve comprising sealing means in form of a thin membrane.

25. A method according to claim 23, comprising the further steps of:
providing a suture sleeve, which is formed of an elastic material and such that at least all of the through holes except one is formed with a slot in the longitudinal direction of the hole, and at least each of the holes being cut through by slots is provided with sealing means;
deflecting the material around a slot shaped hole to be provided with a lead, for widening the slot and breaking the sealing means; and
inserting a lead sideways into the hole through the widened slot.

26. A method according to claim 25, comprising the further steps of:
providing a suture sleeve, of which one hole is unbroken, whereas the other hole or holes each is formed with a slot in the longitudinal direction of the hole;
pre-assembling the suture sleeve with the unbroken hole on one of the leads to be implanted.
